(19) United States
(12) Patent Application Publication
(10) Pub. No.: US 2015/0142134 A1
(43) Pub. Date: May 21, 2015

(54) FOOT FOR MOBILITY DEVICE

(71) Applicant: FlexLeg, LLC, Provo, UT (US)

(72) Inventor: Michael R. Sanders, Provo, UT (US)

(21) Appl. No.: 14/546,870

(22) Filed: Nov. 18, 2014

Related U.S. Application Data
(60) Provisional application No. 61/905,725, filed on Nov. 18, 2013.

(51) Int. Cl. A61F 2/66

(52) U.S. Cl. CPC A61F 2/66; A61F 2002/6614
USPC 623/55

(57) ABSTRACT

A flexible foot for a mobility device may have one or more layers of a resilient material that provides a smoother, more natural walking motion for a user and that is more stable and requires less user energy than a mobility device with a rigid foot. Furthermore, a flexible foot may have a shortened forward toe and extended rear heel, which may provide safety benefits by reducing the risk of falling and incurring further injury.

### CROSS-REFERENCE TO RELATED APPLICATIONS

The present application claims priority and the benefit of U.S. Provisional Patent Application No. 61/905,725, entitled “FOOT FOR MOBILITY DEVICE,” and filed Nov. 18, 2013, the disclosure of which is incorporated herein by reference in its entirety.

### BACKGROUND

Generally, this invention relates to mobility devices. More specifically, the present invention relates to mobility devices that mimic the functionality of a user's lower leg and a device's component in contact with the ground, mimicking the functionality of the user's foot.

Each year, millions of people incur injuries involving the lower leg, ankle, foot, or toe that render their lower leg inoperable during recovery. In 2006, 4.5 million people sought treatment in emergency rooms for such injuries in the United States alone. The lower leg may not be able to bear weight or flex at the knee, at the ankle, or within the foot properly due to these injuries. As a result, the patient's mobility may be severely limited for the duration of the healing process.

During recovery, it is vital for the patient both to maintain mobility as well as keep weight off of the injury to promote rapid and successful healing. Unfortunately, for many people, these are conflicting priorities. In the interest of continuing one's normal daily schedule, some patients may not allow sufficient recovery for their injuries. Others may need to significantly alter their schedules and responsibilities until their injuries properly heal. As a result, various mobility devices have been developed to enhance mobility without detrimentally affecting the healing of the injuries.

Traditional mobility devices provided to patients that have suffered lower leg injuries fall into three primary categories: wheelchairs, crutches, and walking casts. Walking casts are a viable option only once the patient's leg is able to support their body weight. Until that point, wheeled devices or crutches may be used. However, wheelchairs and crutches both require extensive use of the individual's upper body and, in some case, significant upper body strength. Because these devices require the user's hands and upper body to function, in may be difficult to open doors, carry objects, or even use a cellular phone while using a wheelchair or crutches to move.

Furthermore, most patients' daily lives are also not able to accommodate such devices. For example, many homes are not wheelchair accessible or may have long flights of stairs that are difficult to navigate on crutches. The terrain that an individual may need to traverse in their daily schedule may be uneven terrain or have loose surfaces, such as gravel, that may not be conducive to wheeled vehicles or may require significant upper body strength in order to move up a particular slope. The spaces through which an individual may need to pass may not be large enough to accommodate a mobility scooter, or the transport an individual needs to use may not be able to store crutches, such as the bins on an airplane. Therefore, a patient's mobility may remain limited even with the aid of these devices. It would be desirable to provide a patient with a compact device that most accurate replicates the size and movement capability of their own leg. Furthermore, it would be desirable to appropriately replicate the stability and energy transfer of a patient's foot.

Thus, there are a number of problems with mobility devices that can be addressed.

### BRIEF SUMMARY OF THE DISCLOSURE

Implementations of the present disclosure address one or more of the foregoing or other problems in the art with apparatuses, systems, and methods for providing patient's with greater degree of mobility while recovering from a lower leg injury.

In particular, a foot for a mobility device may comprise a resilient member that has a sole affixed to its bottom. The foot may be configured to connect to the mobility device at a point in the forward half of the foot, providing a heel that is longer than a toe. The foot may further comprise additional resilient members disposed on top of the first resilient member to adjust the strength or rate of the foot's flex response.

Additional features of implementations of the disclosure will be set forth in the description which follows, and in part will be obvious from the description, or may be learned by the practice of such implementations. The features of such implementations may be realized and obtained by means of the instruments and combinations particularly pointed out in the appended claims. These and other features will become more fully apparent from the following description and appended claims, or may be learned by the practice of such implementations as set forth hereinafter.

### BRIEF DESCRIPTION OF THE DRAWINGS

In order to describe the manner in which the above-recited and other advantages and features of the disclosure can be obtained, a more particular description of the disclosure briefly described above will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that these drawings depict only typical embodiments of the disclosure and are not therefore to be considered to be limiting of its scope, the disclosure will be described and explained with additional specificity and detail through the use of the accompanying drawings in which:

FIG. 1 is a side view of a foot according to the present disclosure connected to a mobility device.

FIG. 2 is a perspective view of a foot according to the present disclosure.

FIG. 3 depicts a foot according to the present disclosure in use.

FIG. 4 is an exploded view of a foot according to the present disclosure.

FIGS. 5A-B depict an increased force in response to increased flexion of a foot according to the present disclosure.

### DETAILED DESCRIPTION OF THE PREFERRED EMBODIMENTS

One or more implementations of the present disclosure may relate to an apparatus, system, or method of providing increased mobility to patients with lower leg injuries. In particular, one or more implementations of the present disclosure may relate to an apparatus, system, or method of providing increased mobility to patients with lower leg injuries by improving ambulatory stability and energy transfer.

Examples of mobility devices that can provide mobility of similar capability to a patient's own leg are mobility devices such as those disclosed in U.S. patent application Ser. No. 13/856,391, filed Apr. 3, 2013, and entitled MOBILITY DEVICE, U.S. patent application Ser. No. 13/631,741, filed Sep. 28, 2012, and entitled MOBILITY DEVICE, U.S. Provisional Patent Application No. 61/540,938, filed Sep. 29, 2011, and entitled BIO-MIMIC MOBILITY AID, U.S. Provisional Patent Application No. 61/617,458, filed Mar. 29, 2012, and entitled MOBILITY DEVICE, U.S. Provisional Patent Application No. 61/664,660, filed Jun. 26, 2012, and entitled MOBILITY DEVICE, International Application No. PCT/US2012/58160, filed Sep. 29, 2012, and entitled MOBILITY DEVICE, which are herein incorporated by reference.

On such a mobility device, the user secures their leg in the device by use of a plurality of attachment assemblies. In an embodiment, the user may secure their leg to the device using straps comprising hook-and-loop fasteners. The device may include at least one strap on the upper leg and at least one strap on the lower leg. When secured to the device, the user's leg is bent at least partially backwards. A lower leg support allows the user to transfer weight from their upper leg to the ground via the lower leg support and a main body of the mobility device, without relying on their lower leg to bear any of the weight.

The weight transfer occurs from the lower leg support to the main body of the device through an adjustable connection therebetween. The user may alter an angle of the lower leg support such that the user may efficiently transfer weight to the device but also accommodate any difficulties in bending their knee. After the transfer of weight and energy from the user's leg to the main body of the device, the main body transfers that weight and energy to the ground through an artificial foot connected to the bottom of the mobility device. This foot mimics a user's foot to provide improved stability, traction, and energy transfer.

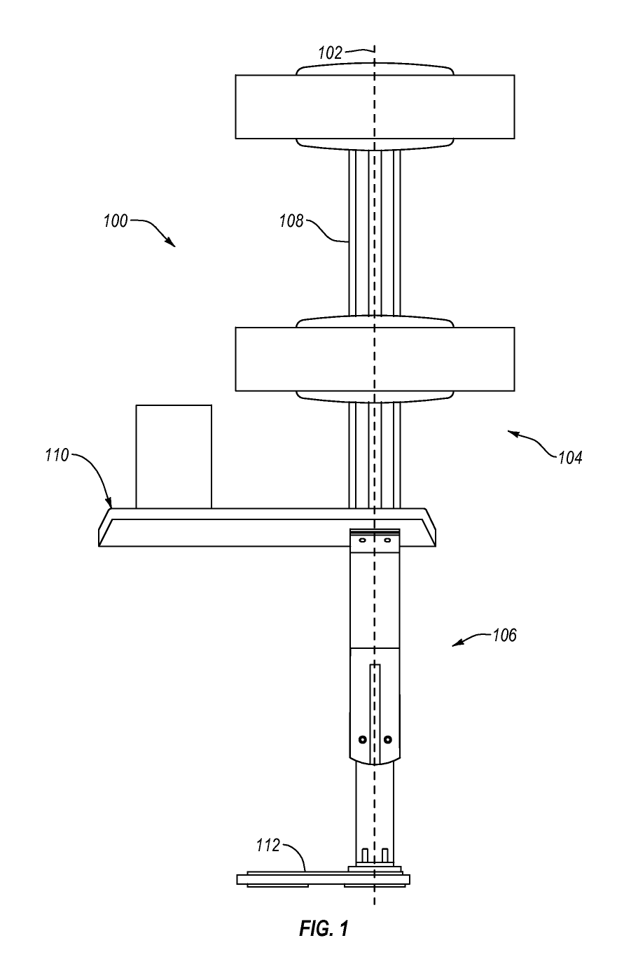

As shown in FIG. 1, the foot 112 attaches to the base of a mobility device 100 in a substantially perpendicular orientation to a weight bearing axis 102 of the mobility device 100. The device has an upper portion 104 and a lower portion 106. The upper portion 104 includes an upper leg support 108 to secure the user's upper leg to the mobility device 100. The mobility device 100 also has a lower leg support 110 adjustably connected to the bottom of the upper portion 104 or the top of the lower portion 106. The upper portion 104 and lower portion 106 may each be independently adjustable in height to accommodate users of varying morphologies. For example, two users of equal height may have different proportions leading to different leg lengths. Further, even users with equal leg lengths may have different femur lengths leading to different knee heights. Independent height adjustments allow for the proper sizing of the mobility device 100 to the user and ensure proper contact between the foot 112 of the device and the ground.

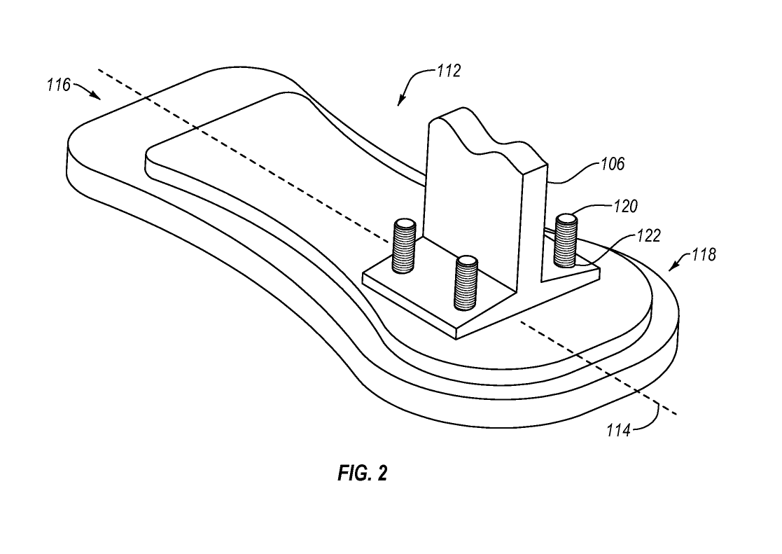

FIG. 2 depicts a close-up of an embodiment of the foot 112 according to the present disclosure. The foot 112 may be symmetrical about a longitudinal center line 114. A symmetric foot 112 on the mobility device 100 enables use of the device 100 on either of a user's legs, as well as predictable movement and stability during use of the mobility device 100. A non-symmetric foot 112 may help provide stability by biasing a user's balance transversely toward a user's center of gravity. The mobility device 100 may be modified to accommodate a user's opposing leg when using a non-symmetric foot 112. In some embodiments, the non-symmetric foot 112 may be removed from the mobility device 100, a tread may be removed from a bottom layer of the foot 112, the foot 112 may be rotated 180° about a longitudinal axis, the tread may be reapplied, and the foot 112 secured to the mobility device 100 again. In other embodiments, the non-symmetric foot 112 may be removed from the mobility device 100, the foot 112 may be rotated 180° about a vertical axis, and the foot 112 secured to the mobility device 100 again. In further embodiments, the foot 112 may have tread on two surfaces allowing the foot 112 to be removed from the mobility device, rotated about one or more axes to be oriented in a desired position, and secured to the mobility device 100.

While the foot 112 is intended to mimic the function of the user's foot, when the foot 112 is connected to the device 100, the foot 112 may extend rearward relative to a user's stride. The portion of the foot 112 that extends rearward is a heel 116 of the foot 112. The foot 112 may be flexible, such that the foot 112 may flex as necessary to accommodate a user's stride, or be rigid with a predetermined curve. The following examples will discuss, without limitation, a flexible version, but similar behavior may be achieved with a rigid foot with a predetermined curve. When the foot 112 is flexible, a predetermined curve is not needed, but may be used.

The use of a layered foot may reduce costs during manufacturing of the foot 112. For example, a layered plastic foot may provide similar properties to a carbon fiber foot with greatly reduced material and production costs. In other mobility devices, the production cost of a carbon fiber foot may account for up to one half of the total production cost of the mobility device. The modularity of a layered foot may provide similar or enhanced performance as compared to the performance of a carbon fiber foot with an associated decrease in cost to manufacture the foot, and therefore a reduction in overall cost of the mobility device, which may result in a lower cost to the user.

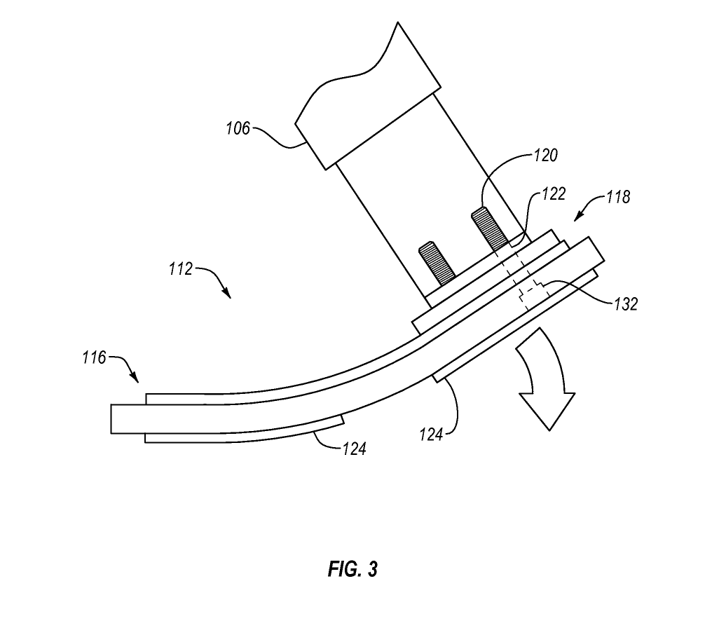

The heel 116 may act as a shock absorber for the mobility device 100. By reducing the vibration upon impact with the ground, the heel 116 provides a more stable contact with the ground. In addition, while flexed, such as in FIG. 3, the heel 116 may apply a force on the ground and on the connection with the lower portion 106 of the mobility device 100 in response to the flexion of the heel 116. The force applied to the lower portion 106 may apply torque on the mobility device 100, pushing the user forward. The amount of force, and therefore torque, applied to the mobility device 100 when the foot 112 is flexed in contact with the ground may be at least partially dependent on the degree of flex experienced by the foot 112. The amount of force applied to the mobility device 100 when the foot 112 is flexed may be at least partially dependent on the material composition of the foot 112.

The force applied on the mobility device 100 by the foot 112 may act as a restoring force, urging the mobility device 100 and user forward. The restoring force may reduce the amount of energy expended by the user while walking with the aid of the mobility device 100, but only applying such a force to restore the mobility device 100 to a substantially upright position after flexion of the foot 112. For example, the foot 112 may apply a force and an associated torque on the mobility device 100 when a user's stride contacts the ground, as depicted in FIG. 3. The resulting flex in the foot 112 may urge the mobility device 100 forward and toward a substantially upright position. The force applied to the mobility device 100 may be at least partially dependent upon the degree of flex of the foot 112. As a result, the force may reduce as the mobility device 100 reaches a substantially vertical position. Furthermore, the force applied to mobility device 100 may reduce to zero as the mobility device 100 attains a substantially vertical position. By applying a substantially forward force only when flexed, the foot 112 may apply a restoring force and assist the user in operating the mobility device 100 while walking. The foot 112 may include one or more connection points 132 that allow the foot 112 to connect to the lower portion 106. Additionally, the heel 116 extending rearward from the connection point 132 between the foot 112 and the lower portion 106 also helps a user stand more stably by providing a flat surface while standing still. The heel 116 may be defined as the portion of the foot 112 extending rearward from the one or more connection points 132.

The foot 112 may include a toe 118 that extends in a forward direction from the one or more connection points 132 in the foot. The toe 118 may be shorter than the heel 116 and may provide little resistance or, in some cases, no resistance to the user when the user begins moving again. The comparatively longer heel 116 may bias the user's balance forward during operation of the mobility device 100. Thus, the foot 112 may partially or fully eliminate an equilibrium point that the user may have to overcome during each stride. Such an equilibrium point may occur, for example, on a foot with a longer toe. While a longer toe may provide an equilibrium point that provides even greater stability when the user is stationary and standing upright, the equilibrium point of a longer toe may also resist movement of the device in a forward direction while walking with the device. A comparatively long heel 116 in combination with a comparatively short toe 118 may provide similar stability when stationary and/or may provide resistance to rearward motion. A comparatively long heel 116 in combination with a comparatively short toe 118 may allow the user to tip the foot 112 forward while walking in a more natural motion without having to overcome an equilibrium point as compared to conventional mobility devices.

The comparatively short toe 118 may also prevent catching the toe 118 on surfaces during use. In a foot 112 with a longer toe 118, the toe 118 may present an unnecessary risk of catching on stairs, curbs, debris or other parts of the terrain a user may need to traverse. A longer toe 118 may inhibit the forward motion of the device during use and may increase the risk of a user falling. Therefore, the short toe 118 of foot 112 may be easier for a new user to operate with a lessened risk of further injury. In some embodiments, the ratio of heel length to toe length may be greater than 2:1. In other embodiments, the ratio of heel length to toe length may be between 2:1 and 6:1. In yet other embodiments, the ratio of heel length to toe length may be 6:1. In further embodiments, the ratio of heel length to toe length may be greater than 6:1.

Still referring to FIG. 3, in an embodiment, the foot 112 may be connected to the lower portion 106 by one or more fasteners 120. In the illustrated embodiment, the fasteners 120 are bolts that connect to complementarily threaded holes 122 in the lower portion 106. The four fasteners 120 may be countersunk in connection points 132 in the foot 112 such that a sole 124 of the foot 112 may maintain traction with the ground without interference from the fasteners 120. The sole 124 may be a single piece affixed to the bottom of the foot 112, or more than one piece affixed to the bottom of foot 112 such that the sole 124 does not substantially inhibit the flex of the foot 112. The foot 112 may be attached to the lower portion 106 by an adhesive, hook-and-loop fasteners, clasps, clamps, magnets, any other appropriate connection, or combinations thereof. A connection between the foot 112 and the lower portion 106 may be removable, but need not be.

Also visible in FIG. 3, the fasteners 120 extend beyond the threaded holes 122 in the lower portion 106. In an embodiment, the foot 112 may comprise a single layer of material to form the heel 116 and toe 118. In another embodiment, the foot 112 may comprise more than one layer of material. The fasteners 120 may have a length sufficient to extend through and affix multiple additional layers to the lower portion 106. Each layer acts like an additional leaf spring in the foot 112 providing additional torque, as mentioned earlier, to cushion a user's step and propel the user forward during a typical stride.

The thickness and number of layers used in the foot 112 may be adjusted to provide for the appropriate force based on a user's size and stride length. In addition, the different layers may comprise different materials. The layers may each comprise a single material or more than one material. In an embodiment, a layer may comprise a single, resilient material. In another embodiment, a layer may comprise two or more pieces of different materials laminated together or stacked upon one another. For example, it may be desirable to have a more elastic material laminated on top of a less elastic material. In an embodiment, the each layer may comprise one or more of a metal; a plastic, such as an injection molded plastic or a fiber reinforced plastic; a carbon fiber; or a polymer.

In order to provide support on a variety of surfaces, the foot 112 may be as wide in the heel 116 as the portion near the fasteners 120 to provide sufficient torsional rigidity. As shown in FIG. 2, in other embodiments, the foot 112 may taper from side to side in the center of the heel 116 and then widen again near the rear of the heel 116. This tapering may allow for a greater degree of longitudinal flex and, therefore, a more comfortable stride for the user. Widening the foot 112 as the heel 116 continues rearward, however, increases rigidity at the rear of the heel 116 and, in at least one embodiment, may aid in stability. In some embodiments, the foot 112 near the rear of the heel 116 may be about the same width as the portion of the foot 112 near the fasteners 120. In other embodiments, the foot 112 near the rear of the heel 116 may be wider than the portion of the foot 112 near the fasteners 120. In yet another embodiment, the foot 112 near the heel 116 may be narrower than the portion of the foot 112 near the fasteners 120.

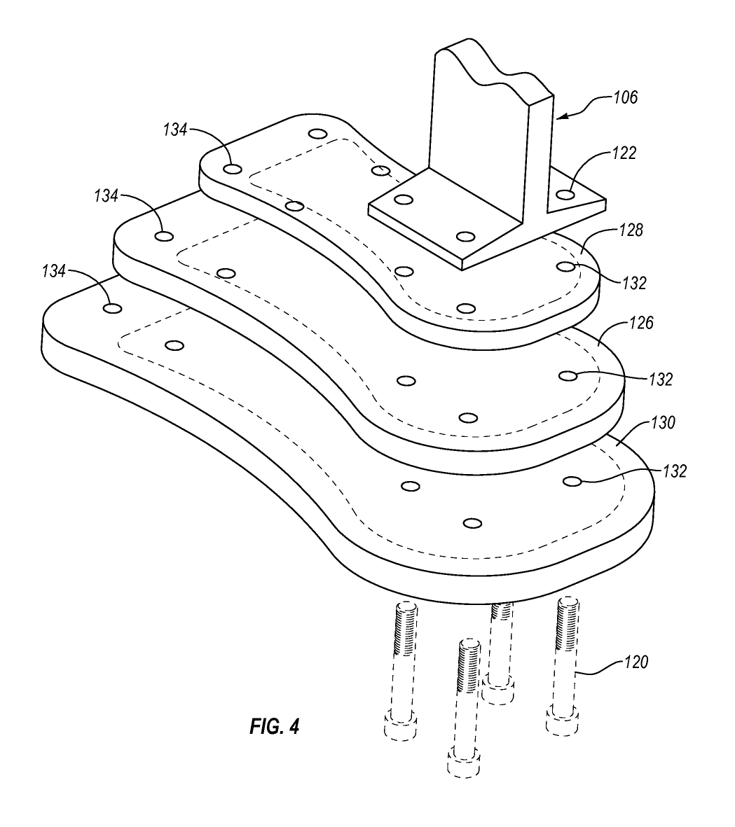

As shown in FIG. 4, a second layer 126 or third layer 128 of material in the foot 112 may be of smaller proportions than the first layer 130 of the foot 112. Alternatively, any additional layers may be of identical proportions to the first layer 130 of the foot 112. Identical proportions may allow all of the layers to be fully interchangeable during use or for repair purposes, thereby increasing the useful lifespan of the foot 112. Forming the additional layers into reduced proportions, however, may ensure the second 126 or third layers 128 do not slide beyond the perimeter of the first layer 130 when the foot 112 is flexed. Additional layers of reduced proportions therefore may reduce wear and prevent damage to the second 126 and third layers 128. Additional layers of reduces proportions may also allow for finer adjustment of the flex of the foot 112 and associated torque and restoring force applied to the lower portion 106.

The layers of the foot 112, including the first layer 130 and/or the additional layers such as the second layer 126 and third layer 128 may include one or more secondary connection points 134. The secondary connection points 134 may allow the foot 112 to be inverted relative to the lower portion 106 and/or allow for other connection configurations using the same fasteners 120 or other fasteners. For example, in an embodiment of a foot 112 that tapers toward the rear of the heel 116 described in relation to FIG. 3, the one or more secondary connection points 134 may allow the lower portion 106 to connect to the foot 112 at the one or more secondary connection points 134 to provide a different flex pattern. In at least one embodiment, the one or more secondary connection points 134 may allow the foot 112 to be turned 180° (i.e., front-to-back) to distribute wear on the foot 112 and enhance the operational lifetime of the foot 112.

The foot 112 for a mobility device 100 may be made by layering one or more layers to form the foot 112. The layers may each be a resilient member of the same or different compositions. The layers may be fastened to one another, and then a fastener may be affixed to enable connection of the foot to a mobility device. The layers may be fastened to one another partially or solely by the one or more fasteners (i.e., fastener 120) that enable connection with a mobility device.

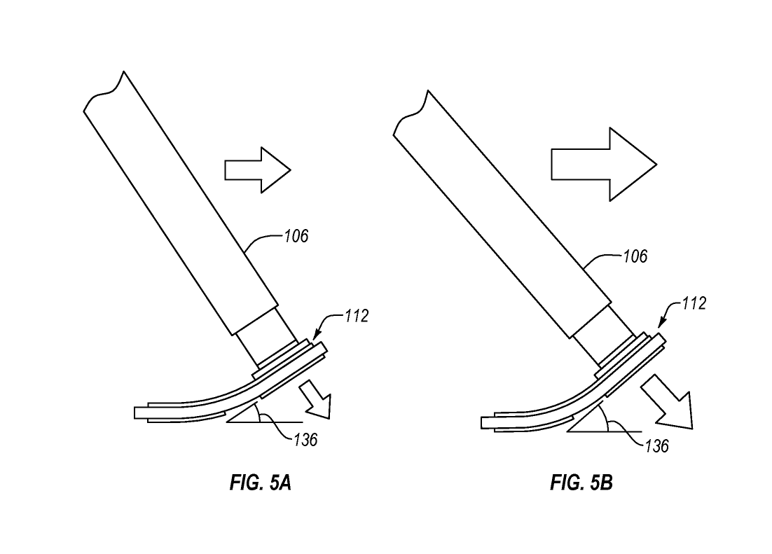

Variations in stride between users may require fine adjustments of the flex response of the foot 112. The restoring force applied by the foot 112 in response to flex may increase with an increasing amount of flex on the foot 112. The amount of flex may be at least partially dependent on the weight of the user and on the user's gait. As shown in FIGS. 5A and 5B, a longer stride (FIG. 5B) may cause the foot 112 to contact the ground at a greater angle than a shorter stride (FIG. 5A). The foot 112 may act like a leaf spring, storing potential energy when flexed and releasing the potential energy as work applied to move the mobility device and user forward while walking. A greater angle of contact with the ground may lead to a greater displacement in the angle 136 of the foot 112, and hence a greater restoring force generated by the foot 112.

When a user is walking on flat terrain, the angle 136 of flex of the foot 112 may be approximately the same as the angle the mobility device 100 from perpendicular to the ground. The angle 136 will change more rapidly for a user of smaller stature. Due to a shorter distance traveled per stride, the angle of the mobility device 100 with the ground and the angle 136 of flex of the foot 112, will change more rapidly for a user with shorter legs than a user with longer legs when each walk at the same linear speed. However, a user of smaller stature will likely weigh less, as well, applying less force to the foot 112 when the mobility device 100 contacts the ground with each stride. This combination demands finer adjustment of the flex response of foot 112.

The use of additional layers in the foot 112 with smaller or different proportions, such as the second layer 126 and third layer 128 shown in FIG. 4, may enable a graduated flex response as the heel 116 strikes the ground first and the foot rolls through the user's stride, providing a more natural gait and balance for a longer stride length. For example, a foot 112 may include only a first layer 130 such as that shown in FIG. 4. A foot 112 with only first layer 130 may provide a continuous and linear application of force to the mobility device 100 when flexed during a user's stride.

In contrast, a foot 112 may include a second layer 126, such as that shown in FIG. 4. A foot 112 including a first layer 130 and a second layer 126 may have a first, softer flex upon the rearmost portion of the heel 116 contacting the ground during a user's stride and a second, firmer flex upon a more forward portion of heel 116 contacting the ground due to the combined stiffness of the first layer 130 and second layer 126 at the more forward portion of the heel. The combination of the first layer 130 and second layer 126 may allow tuning of the foot 112 such that the force applied to the mobility device 100 helps the user walk with a smooth, natural stride.

Because the foot 112 may be tuned to provide improved stability and energy transfer for strides of differing lengths, a foot according the present disclosure may be used with other stride replicating mobility devices, as well, such as crutches or prosthetic devices.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Any element of an embodiment described herein may be combined with any element of any other embodiment described herein. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A foot for a mobility device comprising:
a first resilient member having a top, a bottom, and a longitudinal axis, the first resilient member being configured to connect to a mobility device with the longitudinal axis generally perpendicular to a weight-bearing axis of the mobility device;
a fastener configured to connect the first resilient member to the mobility device; and
a connection point located in a forward half of the first resilient member and defining a toe of the first resilient member extending forward from the connection point and a heel extending rearward of the connection point, wherein the first resilient member is configured to connect to the mobility device at the connection point.

2. The foot of claim 1, wherein the heel is less than or equal to about six times longer than the toe.

3. The foot of claim 1 further comprising one or more additional resilient members that form additional layers disposed on top of the first resilient member.

4. The foot of claim 3, wherein at least one of the one or more additional resilient members comprises a material that is the same as the first resilient member.

5. The foot of claim 1, wherein the first resilient member is symmetrical about the longitudinal axis.

6. The foot of claim 1, wherein the first resilient member comprises a composite material.

7. The foot of claim 1, wherein the first resilient member comprises a fiber reinforced plastic.

8. A method of manufacturing a foot for a mobility device, comprising:
layering a first resilient member on a bottom surface of a second resilient member;
fastening the first resilient member to the second resilient member to form a resilient foot; and
affixing a fastener to the resilient foot, the fastener configured to fasten the first resilient member and second resilient member to a mobility device.

9. The method of claim 8, wherein the first resilient member has a first width and the second resilient member has a second width, the second width being less than the first width.

10. The method of claim 8, wherein the first resilient member has a first length and the second resilient member has a second length, the second length being less than the first length.

11. The method of claim 8, wherein the first resilient member is fastened to the second resilient member with the fastener configured to fasten the first resilient member and second resilient member to a mobility device.

12. The method of claim 8, further comprising layering one or more additional resilient members on a top surface of the second resilient member.

13. The method of claim 8, wherein the fastener configured to fasten the first resilient member and second resilient member to a mobility device is affixed to the resilient foot at a connection point in a forward half of the resilient foot.

14. The method of claim 13, wherein the connection point defines a heel that is greater than or equal to about six times longer than a toe.

15. The method of claim 13, wherein the connection point defines a heel that is less than about six times longer than a toe.

16. A mobility system, the system comprising:
a mobility device having an upper portion and a lower portion, the upper portion configured to selectively attach to a user's leg and the lower portion configured to support a user's weight; and
a foot fastened to the lower portion in an orientation generally perpendicular to a weight bearing axis of the mobility device, the foot comprising
a first resilient member,
a connection point located in a forward half of the first resilient member and defining a toe of the first resilient member extending forward from the connection point and a heel extending rearward of the connection point, wherein the first resilient member is configured to connect to the mobility device at the connection point, and
a fastener that removably connects the first resilient member to the mobility device.

17. The mobility system of claim 16, the foot further comprising a plurality of resilient members.

18. The mobility system of claim 16, wherein the connection point defines a toe and a heel of the foot, the toe extending forward of the connection point and the heel extending rearward of the connection point, wherein a length of the heel is at least twice a length of the toe.

19. The mobility system of claim 16, wherein the foot includes a plastic.

20. The mobility system of claim 16, wherein the foot has a non-linear flex pattern along a length of the foot.
